(19) United States
(12) Patent Application Publication
(10) Pub. No.: US 2024/0252833 A1
(43) Pub. Date: Aug. 1, 2024

(54) METHODS AND SYSTEMS FOR CAPACITOR MAINTENANCE OF AN IMPLANTABLE CARDIOVERTER DEFIBRILLATOR

(71) Applicant: Pacesetter, Inc., Sylmar, CA (US)

(72) Inventors: Xing Pei, Ventura, CA (US); R. Jason Hemphill, Sunset, SC (US); David R. Bowen, Taylors, SC (US); W. Joseph Beauvais, Liberty, SC (US)

(21) Appl. No.: 18/532,500

(22) Filed: Dec. 7, 2023

Related U.S. Application Data
(60) Provisional application No. 63/482,498, filed on Jan. 31, 2023.

(51) Int. Cl. A61N 1/39

(52) U.S. Cl. CPC A61N 1/3937

(57) ABSTRACT

An implantable medical device (IMD) is provided that includes one or more processors and a memory coupled to the one or more processors, wherein the memory stores program instructions. The program instructions are executable by the one or more processors to obtain an initial capacitor maintenance time interval for performing maintenance on a capacitor of the IMD, obtain characteristics of interest related to at least one of the capacitor or the patient, and adjust the initial capacitor maintenance time interval to a first adjusted capacitor maintenance time interval based on the characteristics of interest.

### CROSS-REFERENCE TO RELATED APPLICATIONS

This application claims the benefit of U.S. Provisional Application No. 63/482,498 filed Jan. 31, 2023, titled “METHODS AND SYSTEMS FOR CAPACITOR MAINTENANCE OF AN IMPLANTABLE CARDIOVERTER DEFIBRILLATOR”, the subject matter of which is hereby incorporated by reference in its entirety.

### BACKGROUND

Embodiments of the present disclosure generally relate to methods and systems for determining when to provide maintenance for an implantable medical device (IMD) such as a cardioverter defibrillator (ICD).

IMDs such as implantable cardioverter defibrillator (ICD) devices have advanced to treat cardiac arrhythmia. Device longevity is still a main concern for the healthcare system. A longer device longevity can reduce the risk for the patient to have device change out related issue and reduce the cost for the healthcare system. On the other hand, the charge time is also a competing factor because the charge time is a main factor for episode detection to therapy time. In preventive implantable cardiac defibrillator device systems, the episode detection to therapy time is relatively long as physician opt to allow more opportunity for episode self-termination. Therefore, balancing the three factors, longevity, charge time and time from episode to therapy can improve the system to reduce health care system costs while providing desired therapy to patients.

One component that affects these three factors is the performance of the ICD capacitor. Capacitor/Battery maintenance accounts for up to 40% of the total battery capacity used for the device life. Capacitor maintenance is used for the high voltage device to reform high voltage capacitor to maintenance a relative low charge time. Currently, the capacitor maintenance has either a fixed schedule or a zone (consumption-based window) specific fixed interval.

### SUMMARY

In accordance with embodiments herein, an implantable medical device (IMD) is provided that includes one or more processors and a memory coupled to the one or more processors, wherein the memory stores program instructions. The program instructions are executable by the one or more processors to obtain an initial capacitor maintenance time interval for performing maintenance on a capacitor of the IMD, obtain characteristics of interest related to at least one of the capacitor or the patient, and adjust the initial capacitor maintenance time interval to a first adjusted capacitor maintenance time interval based on the characteristics of interest.

Optionally, the one or more processers are further configured to determine a health index of the capacitor based on the characteristics of interest related to the capacitor and adjust the initial capacitor maintenance time interval to the first adjusted capacitor maintenance time interval based on the characteristics of interest related to the capacitor. In one aspect, the one or more processers are further configured to determine a potential episode of a patient based on the characteristics of interest related to the patient and adjust the first adjusted capacitor maintenance interval to provide a second adjusted capacitor maintenance time interval based on the characteristics of interest related to the patient. In another aspect, the first adjusted capacitor maintenance interval is different than the second adjusted capacitor maintenance interval. In yet another aspect, the one or more processers are further configured to determine a potential episode of a patient based on the characteristics of interest related to the patient and adjust the initial adjusted capacitor maintenance interval to provide the first adjusted capacitor maintenance time interval based on the characteristics of interest related to the patient. In one example, the one or more processers are further configured to perform maintenance on the capacitor when the first adjusted capacitor maintenance time interval is reached. In another example, the one or more processers are further configured to obtain health data related to the capacitor during performance of the maintenance and determine health of the capacitor in response to the maintenance. In yet another example, the one or more processers are further configured to determine a second adjusted capacitor maintenance time interval based on the health data.

In accordance with one or more embodiments herein, a method is provided for performing maintenance on a capacitor of an implantable medical device (IMD). The method includes obtaining and initial capacitor maintenance time interval for performing maintenance on a capacitor of the IMD, obtaining characteristics of interest related to at least one of the capacitor or the patient, and adjusting the initial capacitor maintenance time interval to a first adjusted capacitor maintenance time interval based on the characteristics of interest.

Optionally, the method also includes determining a health index of the capacitor based on the characteristics of interest related to the capacitor and adjusting the initial capacitor maintenance time interval to the first adjusted capacitor maintenance time interval based on the characteristics of interest related to the capacitor. In one aspect, the method additionally includes determining a potential episode of a patient based on the characteristics of interest related to the patient and adjusting the first adjusted capacitor maintenance interval to provide a second adjusted capacitor maintenance time interval based on the characteristics of interest related to the patient. In another aspect, the first adjusted capacitor maintenance interval is different than the second adjusted capacitor maintenance interval. In one example, the method also includes determining a potential episode of a patient based on the characteristics of interest related to the patient and adjusting the initial adjusted capacitor maintenance interval to provide the first adjusted capacitor maintenance time interval based on the characteristics of interest related to the patient. In another example, the method also includes performing maintenance on the capacitor when the first adjusted capacitor maintenance time interval is reached. In yet another example, the method additionally includes obtaining health data related to the capacitor during performance of the maintenance and determining health of the capacitor in response to the maintenance. In yet another aspect, the method also includes determining a second adjusted capacitor maintenance time interval based on the health data.

In accordance with one or more embodiments herein, a computer program product that comprises a non-transitory computer readable storage medium is provided. The non-transitory computer readable storage medium has computer executable code to obtain an initial capacitor maintenance time interval for performing maintenance on a capacitor of the IMD, obtain characteristics of interest related to at least one of the capacitor or the patient, and adjust the initial capacitor maintenance time interval to a first adjusted capacitor maintenance time interval based on the characteristics of interest.

Optionally, the computer program product also has executable code to determine a health index of the capacitor based on the characteristics of interest related to the capacitor and adjust the initial capacitor maintenance time interval to the first adjusted capacitor maintenance time interval based on the characteristics of interest related to the capacitor. In one aspect, the computer program product additionally has executable code to determine a potential episode of a patient based on the characteristics of interest related to the patient and adjust the first adjusted capacitor maintenance interval to provide a second adjusted capacitor maintenance time interval based on the characteristics of interest related to the patient. In one example, the computer program produce also has executable code to determine a potential episode of a patient based on the characteristics of interest related to the patient and adjust the initial adjusted capacitor maintenance interval to provide the first adjusted capacitor maintenance time interval based on the characteristics of interest related to the patient.

### BRIEF DESCRIPTION OF THE DRAWINGS

FIG. 1 illustrates a graphical representation of an implantable medical device (IMD) that is configured to apply defibrillation therapy in accordance with embodiments herein.

FIG. 2 illustrates a schematic diagram of a portion of the IMD of FIG. 1 in accordance with embodiments herein.

FIG. 3 illustrates a schematic block flow diagram of a method for dynamically determining the capacitor maintenance for an IMD in accordance with embodiments herein.

FIG. 4 illustrates a schematic block flow diagram of a method for dynamically determining the capacitor maintenance for an IMD in accordance with embodiments herein.

FIG. 5 illustrates a graph of a maintenance timeline for a capacitor of an IMD in accordance with embodiments herein.

FIG. 6 illustrates a graph of a maintenance timeline for a capacitor of an IMD in accordance with embodiments herein.

FIG. 7 illustrates a graph of a maintenance timeline for a capacitor of an IMD in accordance with embodiments herein.

FIG. 8 illustrates a graph of a maintenance timeline for a capacitor of an IMD in accordance with embodiments herein.

FIG. 9 illustrates a graph of a maintenance timeline for a capacitor of an IMD in accordance with embodiments herein.

FIG. 10 illustrates a graph of a maintenance timeline for a capacitor of an IMD in accordance with embodiments herein.

FIG. 11 illustrates a graph of a maintenance timeline for a capacitor of an IMD in accordance with embodiments herein.

FIG. 12 illustrates a graph of a maintenance timeline for a capacitor of an IMD in accordance with embodiments herein.

FIG. 13 illustrates a graph of a maintenance timeline for a capacitor of an IMD in accordance with embodiments herein.

FIG. 14 illustrates a graph of a maintenance timeline for a capacitor of an IMD in accordance with embodiments herein.

FIG. 15 illustrates a graph of a maintenance timeline for a capacitor of an IMD in accordance with embodiments herein.

FIG. 16 illustrates a digital healthcare system implemented in accordance with embodiments herein.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

Embodiments may be implemented in connection with one or more implantable medical devices (IMDs). Non-limiting examples of IMDs include one or more implantable leadless monitoring and/or therapy devices, and/or alternative implantable medical devices. For example, the IMD may represent a cardioverter defibrillator, pacemaker, cardioverter, cardiac rhythm management device, defibrillator, leadless pacemaker and the like. For example, leadless implantable medical device (LIMD) can include one or more structural and/or functional aspects of the device(s) described in U.S. Pat. No. 9,216,285 “Leadless Implantable Medical Device Having Removable And Fixed Components” which is hereby incorporated by reference. Additionally or alternatively, the IMD may include one or more structural and/or functional aspects of the device(s) described in U.S. Pat. No. 8,391,980 “Method And System For Identifying A Potential Lead Failure In An Implantable Medical Device” and U.S. Pat. No. 9,232,485 “System And Method For Selectively Communicating With An Implantable Medical Device”, which are hereby incorporated by reference.

Additionally or alternatively, the IMD may be a subcutaneous IMD that includes one or more structural and/or functional aspects of the device(s) described in U.S. Pat. No. 10,765,860, titled “Subcutaneous Implantation Medical Device With Multiple Parasternal-Anterior Electrodes”; U.S. Pat. No. 10,722,704, titled “Implantable Medical Systems And Methods Including Pulse Generators And Leads”; U.S. Pat. No. 11,045,643, titled “Single Site Implantation Methods For Medical Devices Having Multiple Leads”, which are hereby incorporated by reference in their entireties. Further, one or more combinations of IMDs may be utilized from the above incorporated patents and applications in accordance with embodiments herein.

All references, including publications, patent applications and patents, cited herein are hereby incorporated by reference to the same extent as if each reference were individually and specifically indicated to be incorporated by reference and were set forth in its entirety herein.

### Terms

The terms “cardiac activity signal,” “cardiac activity signals,” “CA signal” and “CA signals” (collectively “CA signals”) are used interchangeably throughout to refer to measured signals indicative of cardiac activity by a region or chamber of interest. For example, the CA signals may be indicative of impedance, electrical or mechanical activity by one or more chambers (e.g., left or right ventricle, left or right atrium) of the heart and/or by a local region within the heart (e.g., impedance, electrical or mechanical activity at the AV node, along the septal wall, within the left or right bundle branch, within the purkinje fibers). The cardiac activity may be normal/healthy or abnormal/arrhythmic. An example of CA signals includes EGM signals. Electrical based CA signals refer to an analog or digital electrical signal recorded by two or more electrodes, where the electrical signals are indicative of cardiac activity.

The term “COI” refers to a character of interest. Characteristics of interest can include data, information, signals, measurements, parameters, calculations, determinations, or the like. Characteristics of interest can include characteristics of interest of an IMD, ICD, capacitor, patent, or the like. Nonlimiting examples of characteristics of interest include capacitor characteristics of interest such as charge time, voltage decay, maintenance performed, maintenance date, time since maintenance, number of maintenance performed, etc. Nonlimiting examples of characteristics of interest of a patient include potential episodes, heartrate, cardiac activity signals and morphology thereof, health conditions, patient weight, hereditary history, pre-existing medical conditions, or the like.

The term “health care system” refers to a system that includes equipment for measuring health parameters, and communication pathways from the equipment to secondary devices. The secondary devices may be at the same location as the equipment, or remote from the equipment at a different location. The communication pathways may be wired, wireless, over the air, cellular, in the cloud, etc. In one example, the healthcare system provided may be one of the systems described in U.S. Provisional Pat. App. No. 62/875,870 entitled METHODS DEVICE AND SYSTEMS FOR HOLISTIC INTEGRATED HEALTHCARE PATIENT MANAGEMENT, to Rupinder, filed Jul. 18, 2019, the entire contents of which are incorporated in full herein. Other patents that describe example monitoring systems include U.S. Pat. No. 6,572,557; entitled SYSTEM AND METHOD FOR MONITORING PROGRESSION OF CARDIAC DISEASE STATE USING PHYSIOLOGIC SENSORS, filed Dec. 21, 2000, to Tchou et al.; U.S. Pat. No. 6,480,733 entitled METHOD FOR MONITORING HEART FAILURE filed Dec. 17, 1999, to Turcott; U.S. Pat. No. 7,272,443 entitled SYSTEM AND METHOD FOR PREDICTING A HEART CONDITION BASED ON IMPEDANCE VALUES USING AN IMPLANTABLE MEDICAL DEVICE, filed Dec. 14, 2004, to Min et al; U.S. Pat. No. 7,308,309 entitled DIAGNOSING CARDIAC HEALTH UTILIZING PARAMETER TREND ANALYSIS, filed Jan. 11, 2005, to Koh; and U.S. Pat. No. 6,645,153 entitled SYSTEM AND METHOD FOR EVALUATING RISK OF MORTALITY DUE TO CONGESTIVE HEART FAILURE USING PHYSIOLOGIC SENSORS, filed Feb. 7, 2002, to Kroll et. al., the entire contents of which are incorporated in full herein.

The terms “high-voltage shock” and “HV shock” refer to defibrillation stimulus delivered at an energy level sufficient to terminate a defibrillation episode in a heart, wherein in some embodiments the energy level is defined in Joules to be approximately 40 J or more and/or the energy level is defined in terms of voltage to be approximately 750V or more.

The terms “low voltage shock,” “low voltage stimulation,” “LV shock” and the like, refer to stimulus delivered at an energy level below an MV shock energy level, and above a pacing pulse energy level, wherein the energy level is defined in Joules, maximum charge voltage and/or pulse width. In connection with an IMD having a transvenous lead, the foregoing terms refer to stimulation that has an energy level that is no more than approximately 20V, in some embodiments to be between approximately 5V-15V and in other embodiments, to be between 7V-10V.

The terms “medium-voltage shock” and “MV shock” refer to defibrillation stimulus delivered at an energy level sufficient to terminate a defibrillation episode in a heart, wherein the energy level is defined in Joules, pulse width, and/or maximum charge voltage. A MV shock from an IMD with a transvenous lead will have a different maximum energy and/or charge voltage than an MV shock from a subcutaneous IMD with a subcutaneous lead. In connection with an IMD having a transvenous lead, the terms medium voltage shock and MV shock refer to defibrillation stimulation that has an energy level that is no more than approximately 25 J, and more preferably approximately 15-25 J and/or has a maximum voltage of no more than approximately 500V, preferably between approximately 100-475V and more preferably between approximately 400-475V. In connection with an IMD having a subcutaneous lead (e.g., parasternal or otherwise), the terms medium voltage shock and MV shock refer to defibrillation stimulation that has an energy level that is no more than approximately 40 J, and more preferably approximately 30-40 J, and/or has a maximum voltage of no more than approximately 35 V, preferably between approximately 25-35 V and more preferably between approximately 20-35 V.

The phrase “initial capacitor maintenance time interval” refers to a period of time between either the implant of an original capacitor maintenance or a period of time between consecutive capacitor maintenance. In this manner, when implanted an initial capacitor maintenance time interval is between implant time and the occurrence of the original capacitor maintenance. If that initial capacitor maintenance time interval is adjusted as a result of the health of the capacitor, a potential episode being detected, etc. then the new capacitor maintenance time interval is considered an adjusted capacitor time interval. In addition, from the perspective of any additional adjustments, an adjusted capacitor maintenance time interval can also be considered an initial capacitor maintenance time interval. In particular, the initial capacitor maintenance time interval is any capacitor maintenance time interval that can be adjusted according to the methodologies described herein.

The phrase “adjusted capacitor maintenance time interval” refers to any capacitor maintenance period of time that is adjusted from an initial capacitor maintenance time interval as defined herein to a new capacitor maintenance time interval. As an example, if an initial capacitor maintenance time interval is one hundred days and as a result of methodologies described herein a determination is made that this initial capacitor maintenance should be reduced to ninety days, the ninety-day time interval is considered an adjusted capacitor maintenance time interval. If due to the methodologies described herein, the time period of ninety days is determined to again be adjusted to eighty days, the eighty-day time period is considered an adjusted capacitor maintenance time interval. To this end, the ninety-day time period can be considered a first adjusted capacitor maintenance time interval or an initial capacitor time interval without falling outside the definitions herein.

The term “capacitor health data” shall refer to any and all types of information, signals, or the like conveyed from an ICD to a local or remote external device related to a capacitor. Nonlimiting examples of capacitor health data include capacitor charge time, voltage decay rate, amount of capacitor maintenance, capacitor efficiencies, any data, signals, etc. used to determine a capacitor health index, or the like.

The term “capacitor” shall mean that one or more single physical component is utilized to perform the corresponding operation (e.g., retain a charge, deliver a shock, maintain a defined initial or later voltage). In some configurations two or more physical capacitors are coupled in parallel or series with one another or utilized independently to deliver corresponding portions of a shock. When described in relation to capacitor maintenance, the capacitor maintenance can be related to a single capacitor or multiple capacitors.

The term “obtains” and “obtaining”, as used in connection with data, signals, information and the like, include at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The term “POC” shall mean point-of-care. The term “point-of-care” and “POC,” when used in connection with medical diagnostic testing, shall mean methods and devices configured to provide medical diagnostic testing at or near a time and place of patient care. The time and place of patient care may be at an individual's home, such as when providing “at home” point of care solutions. The time and place of patient care may be at a physician's office or other medical facility, wherein one or more medical diagnostic tests may be performed on-site at a time of or shortly after a patient visit and collection of a patient sample. The POC may implement the methods, devices and systems described in one or more of the following publications, all of which are expressly incorporated herein by reference in their entireties: U.S. Pat. No. 6,786,874, entitled “APPARATUS AND METHOD FOR THE COLLECTION OF INTERSTITIAL FLUIDS” issued Sep. 7, 2004; U.S. Pat. No. 9,494,578, entitled “SPATIAL ORIENTATION DETERMINATION IN PORTABLE CLINICAL ANALYSIS SYSTEMS” issued Nov. 15, 2016; and U.S. Pat. No. 9,872,641, entitled “METHODS, DEVICES AND SYSTEMS RELATED TO ANALYTE MONITORING” issued Jan. 23, 2018.

The terms “processor,” “a processor,” “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “real-time” refers to a time frame contemporaneous with normal or abnormal episode occurrences. For example, a real-time process or operation would occur during or immediately after (e.g., within minutes or seconds after) a cardiac event, a series of cardiac events, an arrhythmia episode, and the like. For example, the term “real-time” may refer to a time period substantially contemporaneous with an event of interest. The term “real-time,” when used in connection with collecting and/or processing data utilizing an IMD, shall refer to processing operations performed substantially contemporaneous with a physiologic event of interest experienced by a patient. By way of example, in accordance with embodiments herein, cardiac activity signals are analyzed in real time (e.g., during a cardiac event or within a few minutes after the cardiac event).

The term “subcutaneous” shall mean below the skin, but not intravenous. For example, a subcutaneous electrode/lead does not include an electrode/lead located in a chamber of the heart, in a vein on the heart, or in the lateral or posterior branches of the coronary sinus.

The term “IMD” shall mean an implantable medical device. Embodiments may be implemented in connection with one or more implantable medical devices (IMDs). Non-limiting examples of IMDs include one or more neurostimulator devices, implantable leadless monitoring and/or therapy devices, and/or alternative implantable medical devices. For example, the IMD may represent a subcutaneous cardioverter defibrillator, cardiac monitoring device, pacemaker, cardioverter, cardiac rhythm management device, defibrillator, neurostimulator, leadless monitoring device, leadless pacemaker, and the like. The IMD may measure electrical and/or mechanical information. For example, the IMD may include one or more structural and/or functional aspects of the device(s) described in U.S. Pat. No. 9,333,351, entitled “NEUROSTIMULATION METHOD AND SYSTEM TO TREAT APNEA” issued May 10, 2016 and U.S. Pat. No. 9,044,610, entitled “SYSTEM AND METHODS FOR PROVIDING A DISTRIBUTED VIRTUAL STIMULATION CATHODE FOR USE WITH AN IMPLANTABLE NEUROSTIMULATION SYSTEM” issued Jun. 2, 2015, which are hereby incorporated by reference. The IMD may monitor transthoracic impedance, such as implemented by the CorVue algorithm offered by St. Jude Medical. Additionally or alternatively, the IMD may include one or more structural and/or functional aspects of the device(s) described in U.S. Pat. No. 9,216,285, entitled “LEADLESS IMPLANTABLE MEDICAL DEVICE HAVING REMOVABLE AND FIXED COMPONENTS” issued Dec. 22, 2015 and U.S. Pat. No. 8,831,747, entitled “LEADLESS NEUROSTIMULATION DEVICE AND METHOD INCLUDING THE SAME” issued Sep. 9, 2014, which are hereby incorporated by reference. Additionally or alternatively, the IMD may include one or more structural and/or functional aspects of the device(s) described in U.S. Pat. No. 8,391,980, entitled “METHOD AND SYSTEM FOR IDENTIFYING A POTENTIAL LEAD FAILURE IN AN IMPLANTABLE MEDICAL DEVICE” issued Mar. 5, 2013 and U.S. Pat. No. 9,232,485, entitled “SYSTEM AND METHOD FOR SELECTIVELY COMMUNICATING WITH AN IMPLANTABLE MEDICAL DEVICE” issued Jan. 5, 2016, which are hereby incorporated by reference. Additionally or alternatively, the IMD may be a subcutaneous IMD that includes one or more structural and/or functional aspects of the device(s) described in U.S. application Ser. No. 15/973,195, entitled “SUBCUTANEOUS IMPLANTATION MEDICAL DEVICE WITH MULTIPLE PARASTERNAL-ANTERIOR ELECTRODES” filed May 7, 2018; U.S. application Ser. No. 15/973,219, entitled “IMPLANTABLE MEDICAL SYSTEMS AND METHODS INCLUDING PULSE GENERATORS AND LEADS” filed May 7, 2018; U.S. application Ser. No. 15/973,249, entitled “SINGLE SITE IMPLANTATION METHODS FOR MEDICAL DEVICES HAVING MULTIPLE LEADS”, filed May 7, 2018, which are hereby incorporated by reference in their entireties. Further, one or more combinations of IMDs may be utilized from the above incorporated patents and applications in accordance with embodiments herein. Embodiments may be implemented in connection with one or more subcutaneous implantable medical devices (S-IMDs). For example, the S-IMD may include one or more structural and/or functional aspects of the device(s) described in U.S. application Ser. No. 15/973,219, entitled “IMPLANTABLE MEDICAL SYSTEMS AND METHODS INCLUDING PULSE GENERATORS AND LEADS”, filed May 7, 2018; U.S. application Ser. No. 15/973,195, entitled “SUBCUTANEOUS IMPLANTATION MEDICAL DEVICE WITH MULTIPLE PARASTERNAL-ANTERIOR ELECTRODES”, filed May 7, 2018; which are hereby incorporated by reference in their entireties. The IMD may represent a passive device that utilizes an external power source, and/or an active device that includes an internal power source. The IMD may deliver some type of therapy/treatment, provide mechanical circulatory support, and/or merely monitor one or more physiologic characteristics of interest (e.g., PAP, CA signals, impedance, heart sounds).

Additionally or alternatively, embodiments herein may be implemented in connection with an integrated healthcare patient management system or network, such as described in U.S. application Ser. No. 16/930,791, entitled “METHODS, DEVICE AND SYSTEMS FOR HOLISTIC INTEGRATED HEALTHCARE PATIENT MANAGEMENT” filed Jul. 16, 2020, which is incorporated by reference herein in its entirety.

Additionally or alternatively, embodiments herein may be implemented in connection with the methods and systems described in U.S. application Ser. No. 16/869,733, entitled “METHOD AND DEVICE FOR DETECTING RESPIRATION ANOMALY FROM LOW FREQUENCY COMPONENT OF ELECTRICAL CARDIAC ACTIVITY SIGNALS”, filed May 8, 2020, which is incorporated by reference herein in its entirety.

### System Overview

In accordance with new and unique aspects herein, methods and systems are described that dynamically determine when an IMD such as an ICD should undergo capacitor maintenance. As the capacitor technology and circuitry technology advance, it is possible to obtain the health status of the capacitor. A health status index can be used to adjust the capacitor maintenance time interval to enhance the capacitor performance. Additionally, as the medical science advances, contemporary ICD devices have an advance arrythmia detection algorithm that includes several phases, potential arrhythmia detection (then switch to episodal pacing mode if potential arrythmia is detected), arrythmia detection, ventricular therapy preparation (such high voltage charging, when programmed), arrythmia confirmation, and therapy delivery, post therapy delivery arrythmia redetection, etc. In addition, the detection phase is prolonged to reduce inappropriate shock, such as to allow more time for arrythmia self-termination. Consequently, a method is proposed to utilize these advance detection algorithms and the prolonged detection duration, as well as the capacitor health status index, to have an enhanced capacitor maintenance handling to address the need for short charge time for therapy use and the long device longevity with less consumption. With improved capacitor maintenance handling, the longevity can be increased.

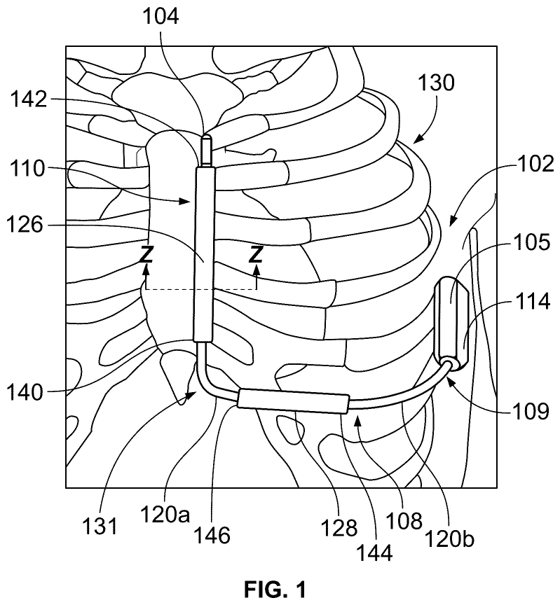

FIG. 1 illustrates a graphical representation of an implantable medical device (IMD) 102 that is configured to apply defibrillation therapy in accordance with embodiments herein. The IMD 102 in the illustrated embodiment is a subcutaneous implantable medical device (SIMD) that is configured to be implanted in a subcutaneous area exterior to the heart. The IMD 102 includes a pulse generator 105 and at least one lead 120a, 120b that is operably coupled to the pulse generator 105. The “at least one lead” is hereinafter referred to as “the lead.” Nevertheless, it should be understood that the term, “the lead,” may mean only a single lead or may mean more than one single lead. The lead 120 includes a lead body that is mechanically connected to the pulse generator 105 and extends from the pulse generator 105 to a distal tip 104 of the lead 120. In some embodiments the lead 120 is an extravascular lead placed in substernal space, and thus is not within blood vessels of the patient. Additionally, the lead 120 can be implanted through the blood vessels into the heart.

The pulse generator 105 includes a housing that contains power circuitry and energy storage devices for generating high-voltage shocks (HV shocks) for defibrillation therapy. The housing may be electrically conductive to form or constitute an electrode utilized to deliver the HV and/or medium-voltage (MV) shocks. The electrode associated with the housing of the pulse generator 105 is referred to as the “CAN” electrode. The pulse generator 105 may be subcutaneously implanted within a pocket at a mix-axillary position along a portion of the ribcage 130 of the patient.

The lead 120 may be subcutaneously implanted. In particular embodiments, the IMD 102 shown is an entirely or fully subcutaneous SIMD. The SIMD may not include a transvenous lead. The lead 120 in the illustrated embodiment includes a first or proximal segment 108 that extends from the pulse generator 105 along an inter-costal area between ribs. The lead 120 has a proximal end 109 that mechanically couples to the pulse generator 105, and electrically connects to the pulse generator 105 to establish conductive path(s) to the electrodes of the lead 120. The proximal segment 108 may be laterally oriented to extend along an anterior axillary area of the ribcage 130. The lead 120 has a second or distal segment 110 that extends from the proximal segment 108 to the distal tip 104. The distal segment 110 may extend along the sternum (e.g., over the sternum or parasternally within one to three centimeters from the sternum). The intersection between the distal and proximal segments 108, 110 may be located proximate to the xiphoid process of the patient.

The lead 120 includes at least one electrode that is electrically connected to the pulse generator 105 and delivers the HV shocks for defibrillation therapy. In the illustrated embodiment, the lead 120 has a first or primary electrode 126 (e.g., sternal coil) disposed along the distal segment 110 and a second or secondary electrode 128 (e.g., transverse coil) disposed along the proximal segment 108. The electrodes 126, 128 may be referred to as shocking electrodes. The electrodes 126, 128 may be elongated coil electrodes. The thickness of the coil electrodes 126, 128 may be in a range from about 3 mm (9 F) to about 10 mm (30 F). In the illustrated embodiment, the primary electrode 126 is longer than the secondary electrode 128. For example, the primary electrode 126 may be about 9 cm, and the secondary electrode 128 may be above 5 cm. In one embodiment, when the pulse generator 105 generates an HV shock, the pulse generator 105 supplies electrical power to both of the electrodes 126, 128. Both electrodes 126, 128 may deliver the HV shocks based on the received electrical power. The electrodes 126, 128 may concurrently deliver the HV shocks to different areas relative to the heart. In other embodiments, the pulse generator 105 can supply electrical power to one or the electrodes 126, 128. In still further embodiments, the pulse generator 105 can supply electrical power to one or both of the electrodes 126, 128 to deliver HV and/or MV shocks that are less than the full shock strength of the associated coil electrode 126, 128.

The electrodes 126, 128 are spaced apart from each other along the length of the lead 120 by a gap segment 131 of the lead body. The gap segment 131 may be proximate to the xiphoid process. The primary electrode 126 may be positioned along an anterior region of the chest, and the secondary electrode 128 may laterally extend between the primary electrode 126 and the pulse generator 105. The electrodes 126, 128 may be subcutaneously positioned at a level that aligns with the heart of the patient for providing a sufficient amount of energy for defibrillation. Although the lead 120 and primary and secondary electrodes 126, 128 are shown positioned outside the ribs, in some embodiments at least one of the primary and secondary electrodes 126, 128 can be interior with respect to the ribs.

The primary electrode 126 may be oriented transverse to an orientation of the secondary electrode 128 when in the implanted position as shown in FIG. 1. For example, the primary electrode 126 has a first orientation extending from a proximal end 140 of the electrode 126 to a distal end 142 of the electrode 126 (defined along the length of the lead 120 relative to the pulse generator 105). The first orientation may be generally parallel to the midsternal line of the patient. The secondary electrode 128 has a second orientation extending from a proximal end 144 of the electrode 128 to a distal end 146 of the electrode 128. The second orientation may be transverse to the first orientation. Optionally, the orientation of the secondary electrode 128 may define an angle between about 60 degrees and 120 degrees (e.g., 70 degrees to 110 degrees) relative to the orientation of the primary electrode 126. Due to the orientation, the lead 120 may be referred to as an L-shaped lead. The primary electrode 126 may be referred to as a parasternal electrode. The secondary electrode 128 may be referred to as a transverse electrode.

In an alternative embodiment, the IMD 102 may lack the secondary electrode 128. For example, the proximal segment 108 may not have any shocking electrodes. The primary electrode 126 may be the only shocking electrode on the lead 120 that delivers the HV shocks supplied from the pulse generator 105.

Optionally, the lead 120 may include one or more sensing electrodes (not shown) for detection of far field electrogram signals. The sensing electrodes may collect subcutaneous cardiac activity (CA) signals in connection with multiple cardiac beats. The IMD 102 may process the CA signals to detect arrhythmias, such as ventricular arrhythmias (VA) (e.g., ventricular tachycardia (VT), ventricular fibrillation, premature ventricular contractions, etc.) and/or atrial fibrillation. If an arrhythmia is detected, the IMD 102 may automatically take one or more actions depending on characteristics of the arrythmia, such as type and severity. The actions may include delivering one or more electrical HV shocks (e.g., shock pulses) via the electrodes 126, 128 in an attempt to achieve cardioversion. Optionally, another IMD may be implanted within the heart, such as a leadless pacemaker. The IMD 102 may be configured to communicate with the other intra-cardiac IMD. For example, the intra-cardiac IMD may signal to the IMD 102 when an arrythmia is detected for the IMD 102 to deliver the HV shocks in response to receiving the signal.

The IMD 102 includes an accelerometer 114 that is used to determine the posture of the patient as further described below. The accelerometer 114 can be within the housing or can of the IMD 102 or external to the housing but located subcutaneously within the patient. In other embodiments, the accelerometer 114 can be included within a separate device that is in communication with the IMD 102. The accelerometer 114 is implanted and/or calibrated in a position and orientation such that, when the patient stands, the accelerometer 114 is located at a reference position and orientation with respect to a global coordinate system that is defined relative to a gravitational direction. For example, the gravitational direction may be along the Z-axis (indicated) while the X-axis is between the left and right arms.

Although the IMD 102 as shown in FIG. 1 includes the lead 120 and electrodes 126 and 128 that are located external to the heart, the embodiments herein can also be incorporated into many other implantable devices, such as transvenous devices, a device located in pectoral regions having one or more lead proximate or into the heart, a leadless device having one or more electrode in physical communication with heart tissue, such as within a ventricle, atrium, and the like.

In some embodiments, the IMD 102 can be, or include capabilities of, a dual-chamber stimulation device capable of treating both fast and slow arrhythmias with stimulation therapy, including cardioversion, defibrillation, anti-tachycardia pacing (ATP) and pacing stimulation, as well as being capable of detecting heart failure, evaluating its severity, tracking the progression thereof, and controlling the delivery of therapy and warnings in response thereto. The IMD 102 may be controlled to sense atrial and ventricular waveforms of interest, discriminate between two or more ventricular waveforms of interest, deliver selected stimulus pulses or shocks based on at least one of a patient's posture, the patient's heart rate, and whether the patient is hemodynamically stable or unstable, and inhibit application of a stimulation pulse to a heart based on the discrimination between the waveforms of interest and the like.

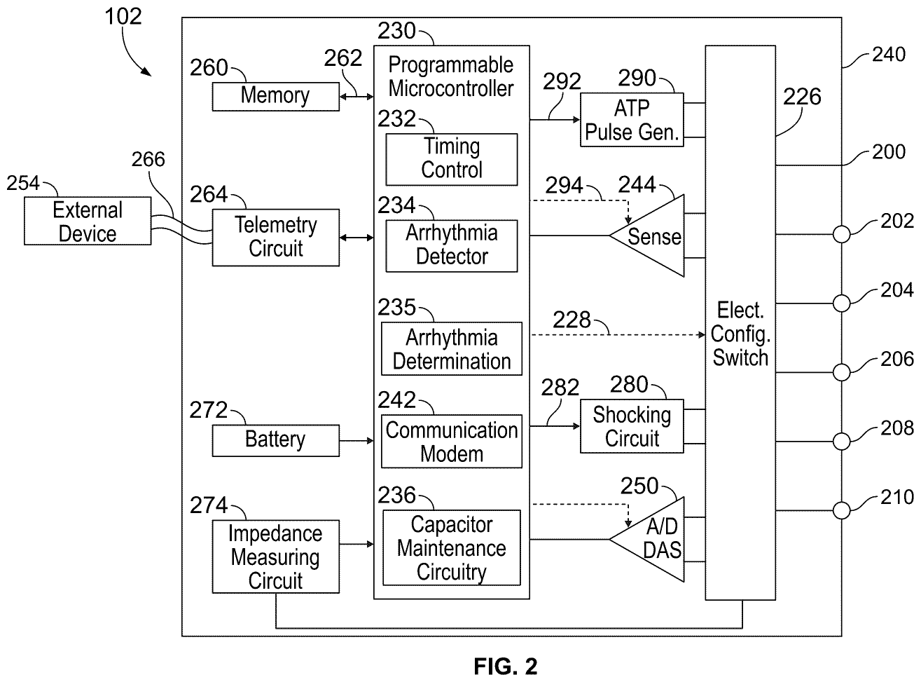

FIG. 2 shows an example block diagram of the IMD 102 formed in accordance with embodiments herein. The IMD 102 may treat both fast and slow arrhythmias, including VA (e.g., further including VT, etc.), with stimulation therapy, including cardioversion, pacing stimulation, suspend tachycardia detection, tachyarrhythmia therapy, and/or the like. In some embodiments, the IMD 102 can be one of an implantable cardioverter defibrillator, pacemaker, cardiac rhythm management device, defibrillator, or leadless pacemaker but is not so limited.

The IMD 102 has a housing 240 to hold the electronic/computing components. The housing 240 (which is often referred to as the “can,” “case,” “encasing,” or “case electrode”) may be programmably selected to function as an electrode for certain sensing modes. Housing 240 further includes a connector (not shown) with at least one terminal 200 and optionally additional terminals 202, 204, 206, 208, 210. The terminals 200, 202, 204, 206, 208, 210 may be coupled to sensing electrodes that are provided upon or immediately adjacent the housing 240. Optionally, more or less than six terminals 200, 202, 204, 206, 208, 210 may be provided in order to support more or less than six sensing electrodes. Additionally or alternatively, the terminals 200, 202, 204, 206, 208, 210 may be connected to one or more leads having one or more electrodes provided thereon, where the electrodes are located in various locations about the heart. The type and location of each electrode may vary. The lead can be positioned in one of a transvenous, subcutaneous, or subxiphoid position. In some embodiments, the IMD 102 can be a subcutaneous IMD coupled to an extravascular lead having a first electrode 126 disposed along a distal segment of the lead and a second electrode 128 disposed along a proximal segment of the lead.

The IMD 102 includes a programmable microcontroller 230 that controls various operations of the IMD 102, including cardiac monitoring. Microcontroller 230 includes a microprocessor (or equivalent control circuitry), RAM and/or ROM memory, logic and timing circuitry 232, state machine circuitry, and I/O circuitry. The timing circuitry 232 can control the timing of the stimulation pulses (e.g., pacing rate, atrio-ventricular (AV) delay, atrial interconduction (A-A) delay, or ventricular interconduction (V-V) delay, etc.). Microcontroller 230 includes an arrhythmia detector 234 that is configured to analyze the cardiac activity (CA) signals over one or more cardiac beats to identify the existence of an arrhythmia. The microcontroller 230 can also include arrhythmia determination circuitry 235 for analyzing the CA signals to assess a presence or absence of R-waves within the cardiac beats. The microcontroller 230 and/or arrhythmia detector 234 can determine a VA episode based on the CA signals. In some embodiments, the microcontroller 230 determines a ventricular arrhythmia based on the absence of at least one R-wave from the cardiac beats.

In other embodiments, the arrhythmia detector 234 and/or arrhythmia determination circuitry 235 can include morphology detection to review and analyze one or more features of the morphology of cardiac signals. The arrhythmia detector 234 and/or arrhythmia determination circuitry 235 analyze the cardiac signals indicative of cardiac events that are sensed by electrodes located proximate to one or more atrial and/or ventricular sites. The cardiac events are sensed over a period of time that includes a detection period that can be followed by an observation period. The cardiac events are analyzed in accordance with conventional ventricular arrhythmia algorithms, such as conventional tachycardia detection algorithms and/or fibrillation detection algorithms. Based on the analysis, the arrhythmia determination circuitry 235 can determine a ventricular arrhythmia episode, such as SVT block, a ventricular tachycardia episode or a ventricular fibrillation episode, etc.

Also, the microcontroller 230 further controls a shocking circuit 280 by way of a control signal 282. The shocking circuit 280 generates shocking pulses that are applied to the heart of the patient to terminate the detected arrhythmia through ATP, less than a full shock strength of one electrode, less than full shock strength with two electrodes, full shock strength with one electrode, full shock strength with two electrodes, etc. The shocking pulses may be selected from the primary electrode 126 and/or the secondary electrode 128 as shown in FIG. 1 or other electrodes/coils discussed herein. In some embodiments the housing 240 may function as an active electrode. The shocking circuit 280 can generate high-voltage and/or medium-voltage and the shocking coils (e.g., electrodes 126, 128) can be configured to deliver high-voltage or medium-voltage shocks.

The microcontroller 230 may also include capacitor maintenance circuitry 236 for determining a capacitor maintenance schedule. The capacitor maintenance schedule includes one or more capacitor maintenance time intervals where the expiration of the time interval results in maintenance of the capacitor. In addition, the capacitor maintenance circuitry can obtain characteristics of interest related to the capacitor or a patient. Characteristics of interest of the capacitor can include capacitor health data such as charge time, voltage decay, maintenance performed, last date of maintenance performed, or the like that can be used to determine the health of the capacitor. In one example, a capacitor health index can be formed, calculated, determined, etc. that provides an indication of the health of the capacitor. Characteristics of interest of the patient can include cardiac activity, heart rate, potential episode, etc. The capacitor maintenance circuitry 236 can then use the characteristics of interest to determine an adjusted capacitor maintenance time interval in real time. The capacitor maintenance circuitry can also continuously dynamically adjust capacitor maintenance intervals in real time as more characteristics of interest are obtained.

A switch 226 is optionally provided to allow selection of different electrode configurations under the control of the microcontroller 230. The electrode configuration switch 226 may include multiple switches for connecting the desired electrodes to the appropriate I/O circuits, thereby facilitating electrode programmability. The switch 226 is controlled by a control signal 228 from the microcontroller 230. Optionally, the switch 226 may be omitted and the I/O circuits directly connected to a housing electrode via terminal 200 and one or more other electrodes via terminals 202, 204, 206, 208, 210.

The IMD 102 is further equipped with a communication modem (modulator/demodulator) 242 to enable wireless communication with other devices, implanted devices, and/or external devices. In one implementation, the communication modem 242 uses high frequency modulation, for example using RF, Bluetooth or Bluetooth Low Energy telemetry protocols. The signals are transmitted in a high frequency range and will travel through the body tissue in fluids without stimulating the heart or being felt by the patient. The communication modem 242 may be implemented in hardware as part of the microcontroller 230, or as software/firmware instructions programmed into and executed by the microcontroller 230. Alternatively, the modem 242 may reside separately from the microcontroller as a standalone component. The modem 242 facilitates data retrieval from a remote monitoring network. The modem 242 enables timely and accurate data transfer directly from the patient to an electronic device utilized by a physician.

The IMD 102 includes sensing circuit 244 selectively coupled to one or more electrodes that perform sensing operations, through the switch 226 to sense cardiac activity data/signals indicative of cardiac activity. The sensing circuit 244 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the features of interest. In one embodiment, switch 226 may be used to determine the sensing polarity of the cardiac signal by selectively closing the appropriate switches.

In the example of FIG. 2, a single sensing circuit 244 is illustrated. Optionally, the IMD 102 may include multiple sensing circuits, similar to sensing circuit 244, where each sensing circuit is coupled to two or more electrodes and controlled by the microcontroller 230 to sense electrical activity detected at the corresponding two or more electrodes. The sensing circuit 244 may operate in a unipolar sensing configuration or a bipolar sensing configuration. Optionally, the sensing circuit 244 may be removed entirely, and the microcontroller 230 performs the operations described herein based upon the CA signals from the A/D data acquisition system 250 directly coupled to the electrodes. The output of the sensing circuit 244 is connected to the microcontroller 230 which, in turn, determines when to store the cardiac activity data of CA signals (digitized by the A/D data acquisition system 250) in a memory 260. In some embodiments, the A/D data acquisition system 250 is coupled to one or more electrodes via the switch 226 to sample cardiac activity signals across any pair of desired electrodes.

By way of example, the external device 254 may represent a bedside monitor installed in a patient's home and utilized to communicate with the IMD 102 while the patient is at home, in bed or asleep. The external device 254 may be a programmer used in the clinic to interrogate the IMD 102, retrieve data and program detection criteria and other features. The external device 254 may be a handheld device (e.g., smartphone, tablet device, laptop computer, smartwatch and the like) that may be coupled over a network (e.g., the Internet) to a remote monitoring service, medical network and the like. The external device 254 may communicate with a telemetry circuit 264 of the IMD 102 through a communication link 266. The external device 254 facilitates access by physicians to patient data as well as permitting the physician to review real-time CA signals while collected by the IMD 102.

The microcontroller 230 is coupled to a memory 260 by a suitable data/address bus 262. The memory 260 stores device parameters associated with each therapy in a collection of VA therapies with different levels of intensity. The levels of intensity for the collection of VA therapies include at least one of i) different energy levels, ii) different types of therapy, or iii) different electrode combination. Further, the collection of VA therapies includes at least one of i) delivering less than a maximal energy with one coil, ii) delivering less than a maximal energy with two coils, iii) delivering maximal energy with one coil, iv) delivering maximal energy with two coils, and v) delivering ATP. The memory 260 stores the acceleration signatures, reference posture data sets, cardiac activity signals, as well as the markers and other data content associated with detection and determination of the arrhythmia.

A battery 272 provides operating power to all of the components in the IMD 102. The battery 272 is capable of operating at low current drains for extended periods of time. The battery 272 also desirably has a predictable discharge characteristic so that elective replacement time may be detected. As one example, the housing 240 employs lithium/silver vanadium oxide batteries. The battery 272 may afford various periods of longevity (e.g., three years or more of device monitoring). In alternate embodiments, the battery 272 could be rechargeable. See, for example, U.S. Pat. No. 7,294,108, titled “Cardiac event micro-recorder and method for implanting same”, which is hereby incorporated by reference.

The IMD 102 further includes an impedance measuring circuit 274, which can be used for many things, including: lead impedance surveillance for proper lead positioning or dislodgement; detecting operable electrodes and automatically switching to an operable pair if dislodgement occurs; measuring thoracic impedance for determining shock thresholds; detecting when the device has been implanted; measuring stroke volume; and detecting the opening of heart valves; and so forth. The impedance measuring circuit 274 is coupled to the switch 226 so that any desired electrode may be used.

The IMD 102 further includes a first chamber pulse generator 290 that generates stimulation pulses for delivery by one or more electrodes coupled thereto. The pulse generator 290 is controlled by the microcontroller 230 via control signal 292. The pulse generator 290 is coupled to the select electrode(s) via the electrode configuration switch 226, which includes multiple switches for connecting the desired electrodes to the appropriate I/O circuits, thereby facilitating electrode programmability.

In some embodiments, the output of the sensing circuitry 244 is connected to the microcontroller 230 which, in turn, triggers or inhibits the pulse generator 290 in response to the absence or presence of cardiac activity. The sensing circuitry 244 receives a control signal 294 from the microcontroller 230 for purposes of controlling the gain, threshold, polarization charge removal circuitry (not shown), and the timing of any blocking circuitry (not shown) coupled to the inputs of the sensing circuitry.

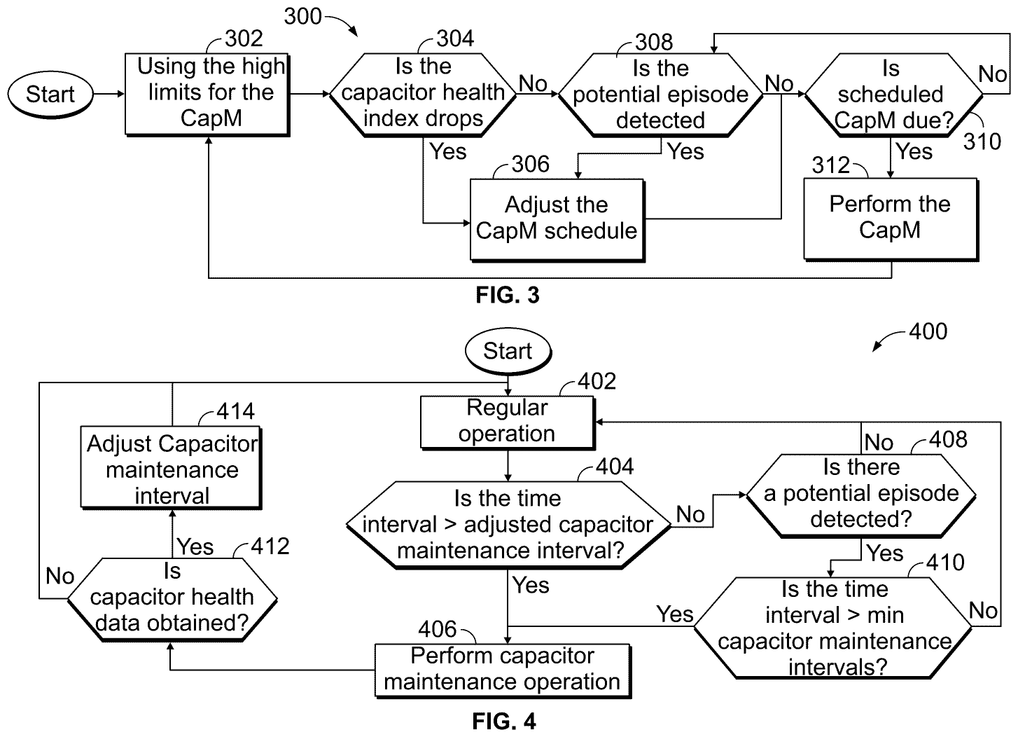

FIGS. 3-4 illustrate methods for dynamically determining the capacitor maintenance for an IMD such as an ICD. The graphs 5-15 illustrate how the method of FIGS. 3-4 can be implemented to improve functioning of a capacitor of an ICD. In examples, the IMD of FIGS. 1-2 is the ICD that performs the methods of FIGS. 3-4.

FIG. 3 illustrates a method 300 of dynamically determining the capacitor maintenance time interval for an IMD such as an ICD. In the method, the capacitor maintenance schedule is determined by setting a limit for a capacitor maintenance schedule and determination of a capacitor health index for adjustment of the capacitor maintenance time interval. Table 1 illustrates an example capacitor maintenance schedule.

TABLE 1

| Items | Low limit | High limit | Access |
|---|---|---|---|
| Optimal CapM 1^{st} region | 1^{st} region min | 1^{st} region max | On shelf to make them equal for |
| (shelf time) | interval | interval | simplicity |
| Optimal CapM 2^{nd} region | 2^{nd} region min | 2^{nd} region max | Actual schedule is between the 2 limits |
| | interval | interval | based on the capacitor health and based |
| Optimal CapM 3^{rd} region | 3^{rd} region min | 3^{rd} region max | on a potential episode detection. |
| | interval | interval | |

The method of FIG. 3 provides a dynamic schedule based on the capacitor health and the episode treatment need (potential episode) that varies between the two limits. At 302, one or more processors operate by utilizing a high limit (as illustrated in Table 1) for capacitor maintenance. In particular, the methodology provides a low limit of the capacitor maintenance (CapM) schedule interval and a high limit of the CapM schedule interval, while the actual CapM schedule interval is dynamic and varies between the two limits or is due right away. By having the low limit and high limit, maintenance can still be provided on a schedule. In this manner, the methodology provides three different CapM trigger events: 1) external user requested CapM, 2) time of CapM is expired, or 3) as a result of a dynamic change related to the capacitor that in one example can be a potential episode being detected and the time CapM is longer than the value of the low limit. In addition, regardless of the functioning of the ICD, capacitor maintenance is performed at the maximum interval.

At 304, the one or more processors determine whether a capacitor health index reduces by a threshold amount. The one more processors, continuously in real-time monitor capacitor health data such as characteristics of interest of the capacitor to determine changes in the capacitor health index of the capacitor. The capacitor health index in one example can be determined by obtaining characteristics of interest related to the capacitor. Characteristics of interest can include charge time of charging to a first phase of CapM, voltage decay rate D1(t) on the capacitor between the end of first phase of CapM and the start of the Turbo phase (top-off recharge of the HV capacitors), charge time of charging to second phase of CapM (Turbo charge) the voltage decay rate D2(t) on the capacitor after the end of second phase of CapM, for N (10) seconds, or the like.

Based on the characteristics of interest of the capacitor, including the capacitor health data, a capacitor health index can be determined in numerous ways. As examples, equations 1 and 2 provide two separate ways to determine or calculate the capacitor health index.

$$cap health index 1 = \frac{Cap voltage 10 sec after CapM 1 st phase completion}{Cap efficiency voltage 10 sec after CapM 1 st phase} \qquad ( 1 )$$

$$cap health index 2 = \frac{Cap voltage 10 sec after completion of 1 st Turbo phase}{Cap efficiency threshold of the CapM 1 st Turbo phase} \qquad ( 2 )$$

The one or more processors continuously in real time determine a cap health index and monitor for a reduction greater than a cap threshold. The cap threshold can be represented by a fraction, percentage, numeral, etc.

If the D1 is faster than the pre-defined D1_threshold and D2 is faster than the pre-defined D2_threshold, the capacitor experienced an extreme deformation, and additional maintenance is needed. After the maintenance, the one or more processors can collect the voltage decay rate D3(t) on the capacitor after the end of maintenance, for a determined period of time N (e.g., 10 seconds). If the D3 is faster than the pre-defined D3_threshold, a clinical alert is generated to indicate a capacitor formation issue. So, if the maintenance is unsuccessful, continued monitoring can be utilized to alert a clinician that the issue with the capacitor is not fixed with and is still present.

When the D1 is slower than the pre-defined D1_threshold or D2 is slower than the pre-defined D2_threshold, a dump can be performed for the CapM, when voltage is reached a pre-defined dump start threshold for evaluate the capacitance, and the dump is terminated when voltage is reached a pre-defined dump end threshold. The time of the dump is recorded, and the effective capacitance is calculated. If the effective capacitance is smaller than the pre-defined threshold, a clinical alert is generated to indicate a capacitor formation issue that the delivered energy may not be sufficient.

When the D1 is slower than the pre-defined D1_threshold or D2 is slower than the pre-defined D2_threshold, and the effective capacitance is within the expectation, the CapM schedule is further refined, in one example as follows:

$${t}_{CapM adjustment} = adjustmenet range * min ( \frac{decrease of cap efficiency}{Cap efficiency range} , 1 ) \\ where adjustmenet range = ( Opt CapM region max interval - Opt CapM region min interval ) , \\ where decrease of cap efficiency = Cap efficiency range upper bound - cap efficiency index ) .$$

In other examples, other nonlinear adjustment can also be used. Further, the t_{CapM} can be further updated with an adjustment (t_{CapM adjustment}): t_{CapM}=t_{CapM}−t_{CapM adjustment}.

If the capacitor health index shows a reduction that is greater than the cap threshold, then at 306, the one or more processors dynamically adjust the CapM schedule. For example, in one example, the low limit may be adjusted, whereas alternatively, the high limit can be adjusted. By adjusting the high limit or low limit, the timing for the capacitor maintenance time interval is changed to reflect a characteristic of interest related to the capacitor. So, as the health of the capacitor deteriorates more rapidly the amount of time before maintenance of the capacitor decreases accordingly.

If the capacitor health index does not reduce past the cap threshold, at 308 the one or more processors determine if a potential episode is detected. In sum, the methodology adjusts capacitor maintenance time interval if the capacitor itself is showing health issues utilizing characteristics of interest of the capacitor itself, or if an indication is presented that the ICD will be in use such that maintenance of the capacitor will be required soon than if an episode where not to occur based on characteristics of interest related to the patient. While FIG. 3 illustrates the capacitor health index as the first item being determined and the potential episode being the second item, these could be exchanged in order, or performed simultaneously without falling outside of the example embodiment. Similar to when the capacitor health index has reduced more than a threshold amount, when a potential episode is detected at 308, the CapM schedule is dynamically adjusted accordingly to address the potential episode.

If at 308, a potential episode is not detected, then the CapM schedule remains, and at 310 the one or more processors determine whether maintenance is due. To this end, after an adjustment to the CapM schedule the determination is similarly made. In particular, if the low limit or high limit are adjusted based on the capacitor health data (index) or the patient health (potential episode), this new schedule is then utilized to determine when capacitor maintenance should occur. Alternatively, if no change is made, then the original low limit and high limit are utilized to determine if maintenance is due. If not, the one or more processors continue monitoring the health of the capacitor and patient to determine if the low limit or high limit should dynamically change. Alternatively, if maintenance is due, then at 312 the capacitor maintenance is performed, and a new low limit and high limit are determined based on the maintenance. By continuously monitoring the capacitor health and patient health, maintenance of the capacitor is dynamically adjusted in real time to provide maintenance only when needed. As a result, the life of the capacitor improves and in turn increases the life of the ICD.

FIG. 4 illustrates an alternative method 400 of dynamically determining the capacitor maintenance time interval for an IMD such as an ICD. In one example, a system that includes the ICD as described in relation to FIGS. 1-2 is utilized to implement the method.

At 402, one or more processors operate an ICD. During such operation, at 404 a determination is made whether a time interval is greater than an initial capacitor maintenance time interval or an adjusted capacitor maintenance time interval. The ICD begins with an initial capacitor maintenance time interval that represents a period of time before maintenance of a capacitor of an ICD is to occur. The methodology as provided by FIG. 3 and this FIG. 4 provide operations for dynamically adjusting the capacitor maintenance time interval from the initial capacitor maintenance time interval to an adjusted capacitor maintenance time interval.

The one or more processors continuously adjusts the time interval for capacitor maintenance by determining if a health condition of the capacitor is deteriorating or if a potential episode can occur to the patient. Based on these characteristics of interest of the capacitor and patient, the time interval is dynamically adjusted during use. Still, when initially implanted, the capacitor can still include an initial capacitor maintenance time interval if no adjustment is determined to be required. If at 404, the time interval is greater than the interval for capacitor maintenance, then at 406, the one or more processors perform capacitor maintenance.

If at 404, the time interval is not greater than the initial capacitor maintenance time interval (e.g., it is not yet time for maintenance), at 408, the one or more processors determine whether a potential episode is detected. In sum, based on characteristics of interest of the patient that can be obtained through measurements of the ICD, or communicated to the ICD, determinations regarding a potential episode are determined as previously described. If at 408 no potential episodes exist, then the one or more processors continue to operate the ICD accordingly. However, if at 408 a potential episode does exist, then at 410, the one or more processors provide an adjusted capacitor maintenance time interval and determine whether the current time interval is greater than the adjusted capacitor maintenance time interval, or minimum, capacitor maintenance time interval. If not, the one or more processors continue operating the ICD until such the adjusted capacitor maintenance time interval is reached. However, if at 408 the adjusted capacitor maintenance time interval is reached, then at 406, the one or more processors perform a capacitor maintenance operation.

After capacitor maintenance is performed, at 412, the one or more processors obtain capacitor health data and determine whether the capacitor maintenance time interval needs to be adjusted based on the health of the capacitor. If not, the one or more processors continue to operate the ICD with a determined, or initial capacitor maintenance time interval. However, if the capacitor is considered not to have sufficient health, then at 414 the capacitor maintenance time interval is adjusted (to an adjusted capacitor maintenance time interval) based on the health of the capacitor. In one example, a capacitor health index is obtained in the process of determining the health of the capacitor.

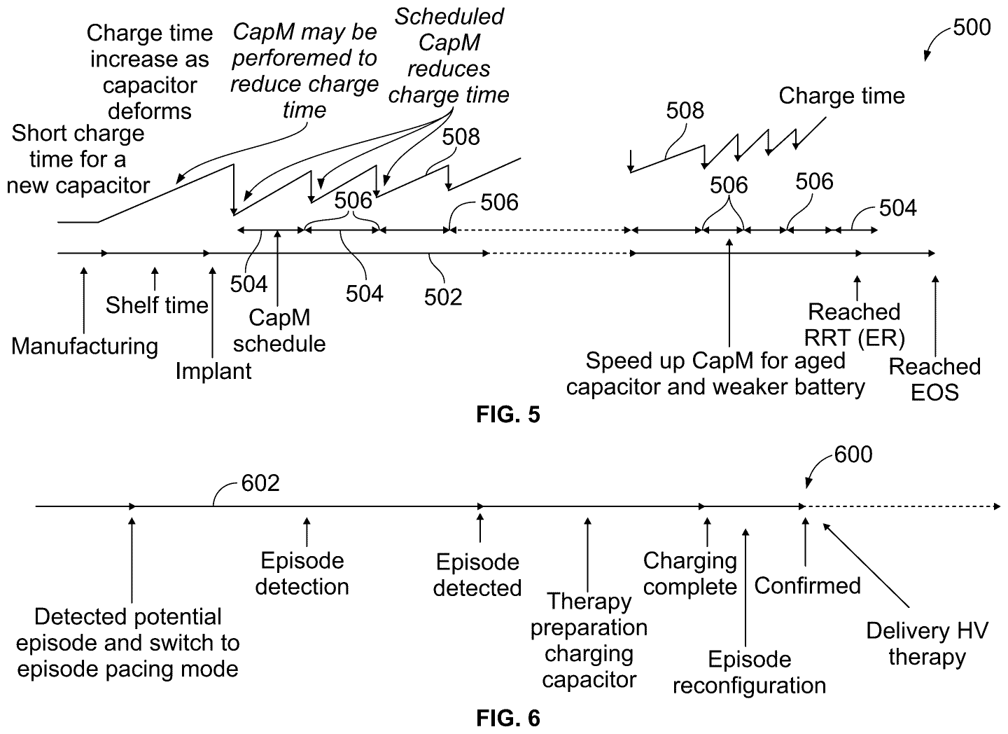

FIGS. 5-15 illustrate numerous graphs that show how the methodology of FIGS. 3 and 4 can be utilized to improve maintenance timing for a capacitor of an ICD.

FIG. 5 illustrates a graph of a capacitor that does not utilize the methodology described by this disclosure, including the methods of FIGS. 3 and 4. The graph 500 shows a timeline 502 that includes manufacturing time, shelf-time and implant. Also provided is the CapM schedule 504 in use that merely provides previously determined time intervals between maintenance 506. Also illustrated on the timeline 502 is the charge time 508 that increases over time until the capacitor maintenance reduces the charge time. As illustrated, once a certain capacitor age is reached, maintenance 506 occurs with more frequency to enhance functionality of the capacitor.

FIG. 6 meanwhile illustrates a graph 600 of the functionality of an ICD that includes ventricular episode detection, episode detection, therapy charging, episode reconfirmation, and therapy delivery over time 602. In contemporary high voltage devices, the detection intervals are set to higher to allow more self-termination of episode to reduce inappropriate high voltage shock. As can be seen from review of the graph of FIG. 6, by simply utilizing a CapM schedule, without consideration of potential episodes, needed capacitor maintenance can be missed.

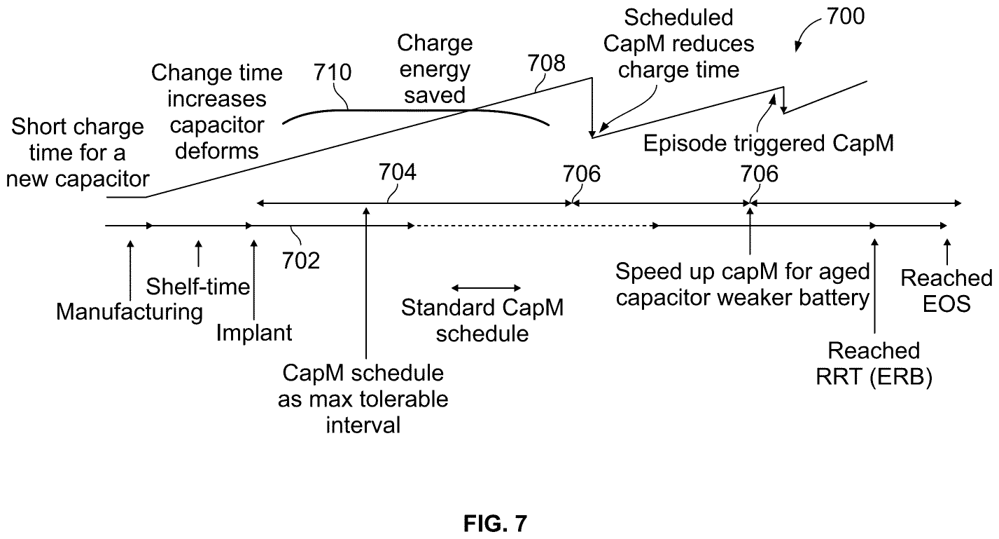

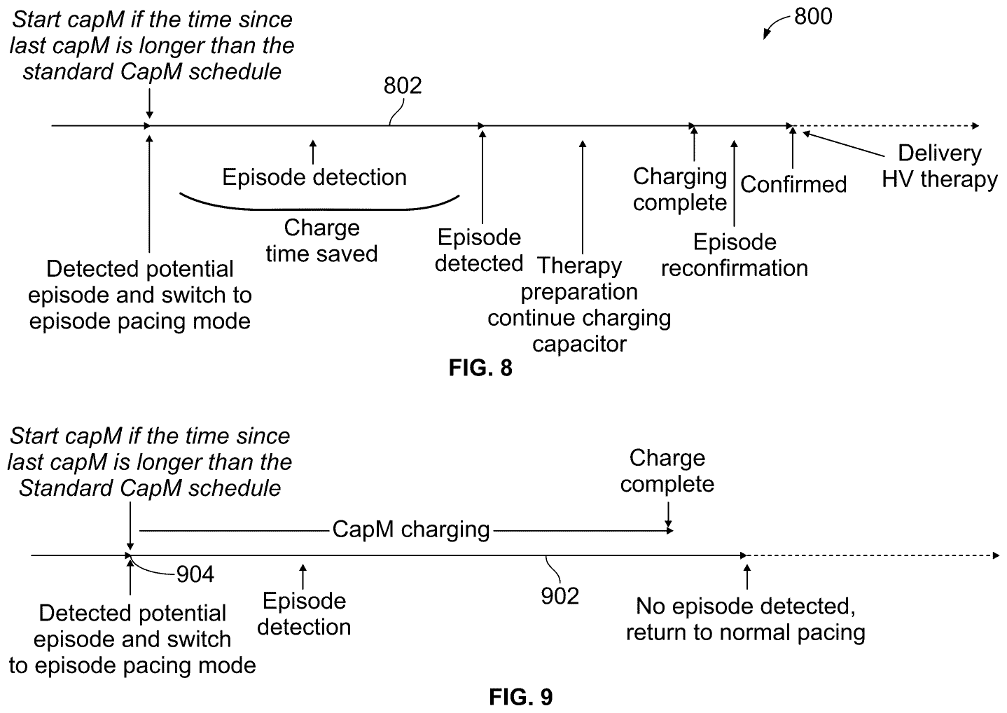

FIGS. 7 and 8 illustrate graphs of a similar capacitor of an ICD and how utilizing the methodologies of FIG. 3 or 4 both saves energy and increases the life of the capacitor. In FIG. 7, again the graph 700 provides a timeline 702 that includes manufacturing time, shelf-time and implant. Here, the CapM schedule 704 is illustrated where the maintenance 706 is dynamically determined in real time based on the capacitor health data and the potential for an episode (as tracked in FIG. 8). As illustrated, the charge time 708 is significantly increased before the first maintenance because the CapM schedule utilizes the maximum tolerable interval when a determination is made that maintenance is not need sooner because of the health of the capacitor or patient. Consequently, by increasing the charge time, charge energy 710 is saved compared to a methodology as seen in FIG. 5 that simply provides a determined time interval for the maintenance. To this end, additional intervals between maintenance are additionally increased, saving energy, until the maintenance is actually needed as a result of capacitor health, or as a result of a potential episode. Therefore, the number of the CapM events is reduced, saving battery and prolonging longevity of the capacitor.

FIG. 8 illustrates how ICD sensing can be performed while charging to provide the methodology herein. The graph 800 illustrates a triggered CapM 802. Here a ventricular episode detection and therapy of a potential episode triggered the CapM. To this end maintenance can be provided when a potential episode is detected to save or reduce charge time prior to therapy.

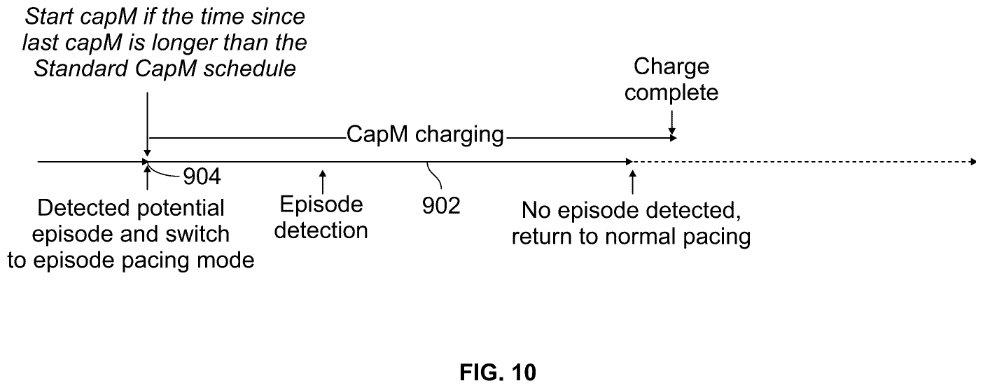

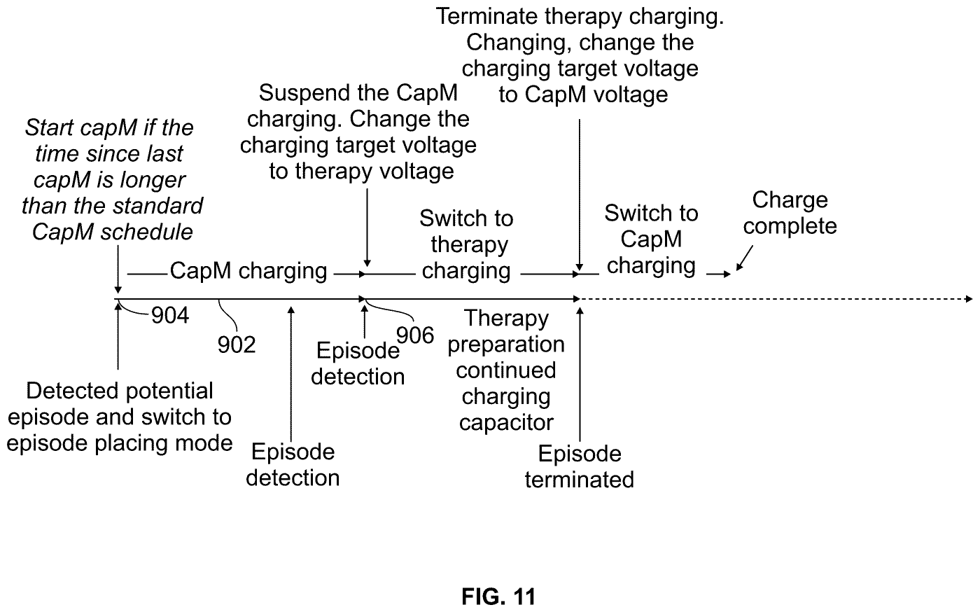

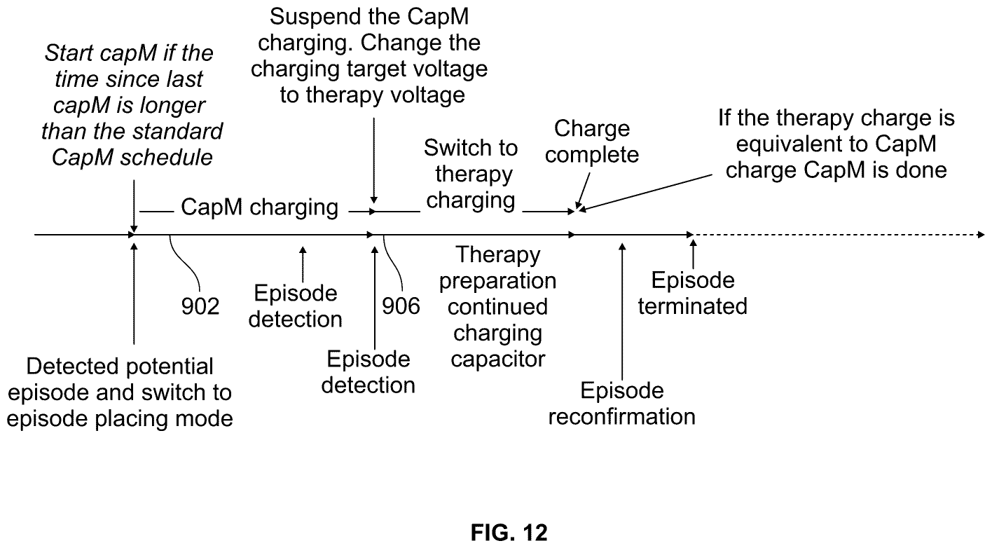

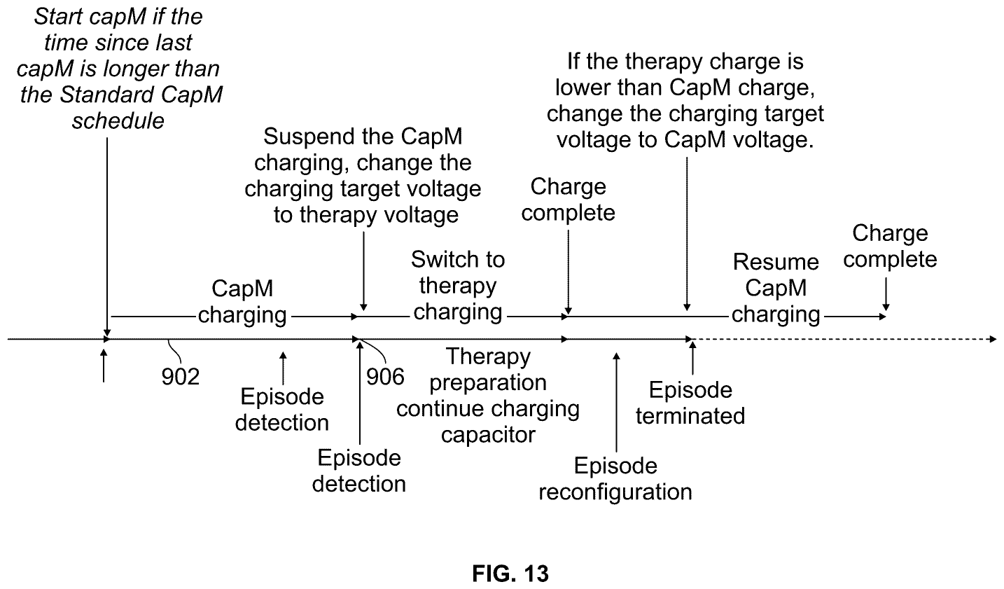

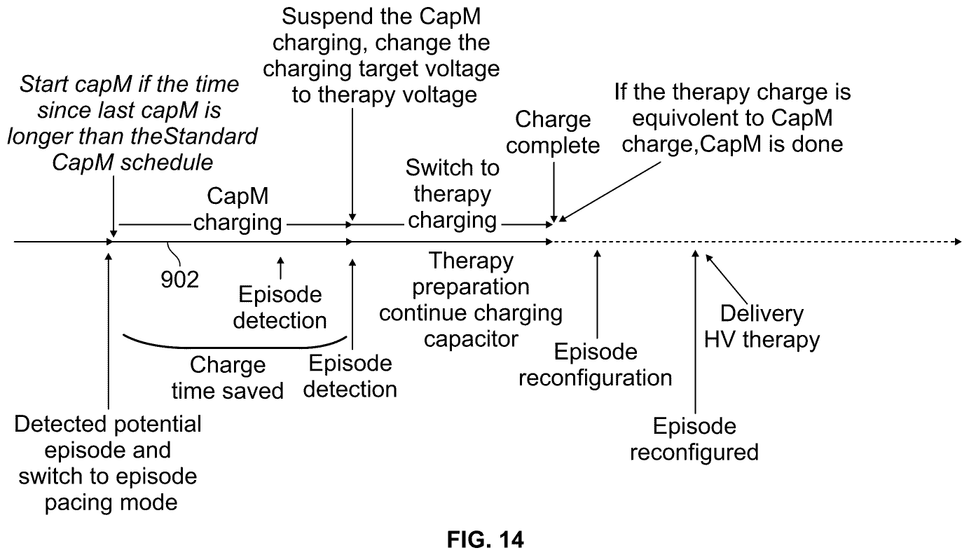

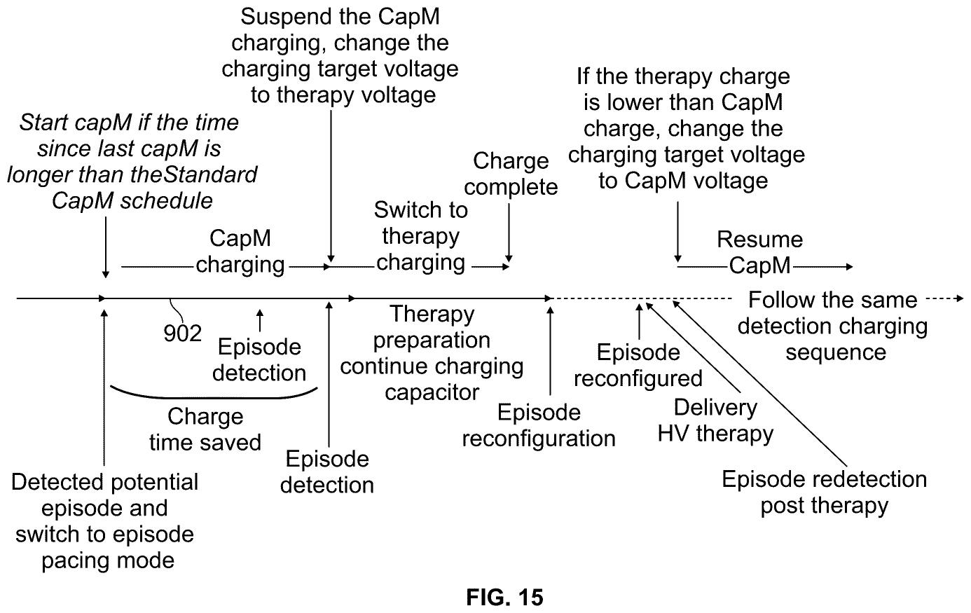

FIGS. 9-15 illustrate numerous examples of potential episode detection during ventricular episode detection and therapy and the effect of CapM charging and CapM time interval adjustment on a timeline 902 as a result of the methodology of FIGS. 3-4. In FIG. 9 a potential episode 904 occurs without an episode later being detected such that charging ends before a return to a normal pacing mode. FIG. 10 illustrates when there is a potential episode without an episode detected later and charging ends after returning to pacing mode. FIG. 11 shows a potential episode determined where an episode 906 is detected, but later self-terminated before therapy charging is complete. Consequently, the CapM time interval remains unchanged. FIG. 12 illustrates a potential episode 904 determined with an episode detected 906, but later self-terminated after therapy charging is complete and before reconfirmation is complete. Here the CapM time interval is complete. FIG. 13 provides a potential episode 904 is determined with the episode detected 906, but later self-terminated after therapy charging is complete, but before reconfirmation is complete. In this case the scheduled CapM time interval is continued. FIG. 14 shows a potential episode determined 904 and an episode detected 906 with therapy delivered and the CapM time interval is complete. FIG. 15 illustrates where a potential episode 904 is determined and the episode is detected 906, therapy is delivered, and CapM time interval restarts. In each instance, CapM time interval adjustments are provided and/or charge time is improved, enhancing the functioning of the ICD.

The method addresses the charge time by charging the capacitor when a potential episode is encountered. The charging during the detection phase reduces the overall charge time to therapy. For example, for a VF episode of 300 ppm (200 ms interval), with a detection number of event interval set to 18 events, the detection time can be 18*200 ms=3600 ms (about 3 and a half seconds)=3.6 second. This is a sizable portion of the overall charge time.

For the programming of this enhanced CapM, the enhanced CapM is only allowed to turn on in the device where the maximum therapy voltage is equal to greater than the capacitor maintenance voltage. This is to address the potential concern that in pediatrics, the maximum therapy voltage is limited to the maximum voltage programmed. Thus, an improved system and method for providing capacitor maintenance is disclosed, increasing life of the capacitor and ICD.

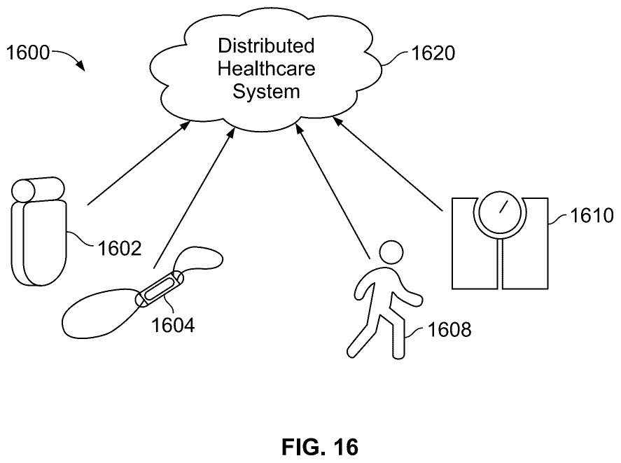

FIG. 16 illustrates a digital healthcare system 1600 implemented in accordance with embodiments herein. The system 1600 obtains characteristics of interest of a capacitor derived from monitoring an ICD, potential episodes, treatment, changes in therapy/medication and the like. The healthcare system 1600 may include wearable devices that communicate with an IMD or accelerometer and a remote database. As a result, the healthcare system 1600 may monitor health parameters of patient, including episodes, applied therapies, etc., and provide a diagnosis for the patient based on the monitored health parameters.

The system 1600 may be implemented with various architectures, which are collectively referred to as a healthcare system 1620. By way of example, the healthcare system 1620 may be implemented as described herein. The healthcare system 1620 is configured to receive data, including ICD data and capacitor health data from a variety of external and implantable sources including, but not limited to, an active ICD 1602 capable of monitoring and delivering therapy to a patient, passive IMDs or sensors 1604, wearable sensors 1608, and point-of-care (POC) devices 1610 (e.g., at home or at a medical facility). Any of the ICD 1602, sensor 1604, and/or sensor 1608 may implement capacitor maintenance circuitry and perform the analysis of capacitor maintenance as described herein. The data from one or more of the external and/or implantable sources is collected and communicated to one or more secure databases within the healthcare system 1620. Optionally, the patient and/or other users may utilize a device, such as a smart phone, tablet device, etc., to enter data. For example, a patient may use a smart phone to provide feedback concerning activities performed by the patient, a patient diet, nutritional supplements and/or medications taken by the patient, how a patient is feeling (e.g., tired, dizzy, weak, good), etc.

### CLOSING

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

Some or all of the Figures herein illustrate various methods and processes implemented in accordance with embodiments herein. The operations herein may be implemented by hardware, firmware, circuitry and/or one or more processors housed partially and/or entirely within an IMD, a local external device, remote server or more generally within a health care system. Optionally, the operations herein may be partially implemented by an IMD and partially implemented by a local external device, remote server or more generally within a health care system. For example, the IMD includes IMD memory and one or more IMD processors, while each of the external devices/systems (ED) (e.g., local, remote or anywhere within the health care system) include ED memory and one or more ED processors.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It should be recognized that, to the extent embodiments herein are described to apply certain mathematical combinations of select variables, the same variables may be combined in other mathematical combinations that are also indicative of the same result. For example, when a single data point is utilized for a particular variable, additionally or alternatively, a mean, average, sum, or other mathematical combination of multiple data points may be utilized for the same variable.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An implantable medical device (IMD) comprising:
one or more processors; and
a memory coupled to the one or more processors, wherein the memory stores program instructions, wherein the program instructions are executable by the one or more processors to:
obtain an initial capacitor maintenance time interval for performing maintenance on a capacitor of the IMD;
obtain characteristics of interest related to at least one of the capacitor or the patient; and
adjust the initial capacitor maintenance time interval to a first adjusted capacitor maintenance time interval based on the characteristics of interest.

2. The IMD of claim 1, wherein the one or more processers are further configured to determine a health index of the capacitor based on the characteristics of interest related to the capacitor and adjust the initial capacitor maintenance time interval to the first adjusted capacitor maintenance time interval based on the characteristics of interest related to the capacitor.

3. The IMD of claim 2, wherein the one or more processers are further configured to determine a potential episode of a patient based on the characteristics of interest related to the patient and adjust the first adjusted capacitor maintenance interval to provide a second adjusted capacitor maintenance time interval based on the characteristics of interest related to the patient.

4. The IMD of claim 3, wherein the first adjusted capacitor maintenance interval is different than the second adjusted capacitor maintenance interval.

5. The IMD of claim 1, wherein the one or more processers are further configured to determine a potential episode of a patient based on the characteristics of interest related to the patient and adjust the initial adjusted capacitor maintenance interval to provide the first adjusted capacitor maintenance time interval based on the characteristics of interest related to the patient.

6. The IMD of claim 1, wherein the one or more processers are further configured to perform maintenance on the capacitor when the first adjusted capacitor maintenance time interval is reached.

7. The IMD of claim 6, wherein the one or more processers are further configured to obtain health data related to the capacitor during performance of the maintenance and determine health of the capacitor in response to the maintenance.

8. The IMD of claim 7, wherein the one or more processers are further configured to determine a second adjusted capacitor maintenance time interval based on the health data.

9. A method for performing maintenance on a capacitor of an implantable medical device (IMD) comprising:
obtaining and initial capacitor maintenance time interval for performing maintenance on a capacitor of the IMD;
obtaining characteristics of interest related to at least one of the capacitor or the patient; and
adjusting the initial capacitor maintenance time interval to a first adjusted capacitor maintenance time interval based on the characteristics of interest.

10. The method of claim 9, further comprising determining a health index of the capacitor based on the characteristics of interest related to the capacitor and adjusting the initial capacitor maintenance time interval to the first adjusted capacitor maintenance time interval based on the characteristics of interest related to the capacitor.

11. The method of claim 10, further comprising determining a potential episode of a patient based on the characteristics of interest related to the patient and adjusting the first adjusted capacitor maintenance interval to provide a second adjusted capacitor maintenance time interval based on the characteristics of interest related to the patient.

12. The method of claim 11 wherein the first adjusted capacitor maintenance interval is different than the second adjusted capacitor maintenance interval.

13. The method of claim 9, further comprising determining a potential episode of a patient based on the characteristics of interest related to the patient and adjusting the initial adjusted capacitor maintenance interval to provide the first adjusted capacitor maintenance time interval based on the characteristics of interest related to the patient.

14. The method of claim 9, further comprising performing maintenance on the capacitor when the first adjusted capacitor maintenance time interval is reached.

15. The method of claim 14, further comprising obtaining health data related to the capacitor during performance of the maintenance and determining health of the capacitor in response to the maintenance.

16. The method of claim 15, further comprising determining a second adjusted capacitor maintenance time interval based on the health data.

17. A computer program product comprising a non-transitory computer readable storage medium comprising computer executable code to:
obtain an initial capacitor maintenance time interval for performing maintenance on a capacitor of an implantable medical device (IMD);
obtain characteristics of interest related to at least one of the capacitor or the patient; and
adjust the initial capacitor maintenance time interval to a first adjusted capacitor maintenance time interval based on the characteristics of interest.

18. The computer program product of claim 17, further configured to determine a health index of the capacitor based on the characteristics of interest related to the capacitor and adjust the initial capacitor maintenance time interval to the first adjusted capacitor maintenance time interval based on the characteristics of interest related to the capacitor.

19. The computer program product of claim 18, further configured to determine a potential episode of a patient based on the characteristics of interest related to the patient and adjust the first adjusted capacitor maintenance interval to provide a second adjusted capacitor maintenance time interval based on the characteristics of interest related to the patient.

20. The computer program product of claim 17, further configured to determine a potential episode of a patient based on the characteristics of interest related to the patient and adjust the initial adjusted capacitor maintenance interval to provide the first adjusted capacitor maintenance time interval based on the characteristics of interest related to the patient.
